(19) United States
(12) Patent Application Publication
(10) Pub. No.: US 2024/0091781 A1
(43) Pub. Date: Mar. 21, 2024

(54) INTEGRATED THERMAL CONDITIONING AND PCR IN A MOLECULAR POC DIAGNOSTIC SYSTEM

(71) Applicant: QUANTUMDX GROUP LIMITED, Newcastle Upon Tyne, Tyne and Wear (GB)

(72) Inventors: Molly JOWSEY; Matthew SELBY

(21) Appl. No.: 18/249,535

(22) PCT Filed: Oct. 14, 2021
(86) PCT No.: PCT/GB2021/052667

§ 371 Date: Apr. 18, 2023

(30) Foreign Application Priority Data
Oct. 19, 2020 (GB) 2016541.1

(51) Int. Cl. B01L 7/00; B01L 3/00; C12Q 1/686

(52) U.S. Cl. CPC B01L 7/525; B01L 3/50273; B01L 3/502738; C12Q 1/686; B01L 2200/0621; B01L 2200/10; B01L 2200/16; B01L 2300/021; B01L 2300/023; B01L 2300/042; B01L 2300/0627; B01L 2300/0816; B01L 2300/1894; B01L 2400/0481

(57) ABSTRACT

Disclosed herein are microfluidic test cassettes or chips that are received within a POC diagnostic device, and that can directly test biological samples that have had no or minimal processing to remove PCR inhibitor. The microfluidic test cassettes or chips allow for on-cassette/on-chip processing within the size confines of the cassette/chip by utilizing a same heating zone for multiple processing steps.

### TECHNICAL FIELD

The present invention relates to integration of molecular testing methodologies onto point of care (POC) diagnostic devices and systems as well as methods of molecular testing based on the same. More particularly, it relates to microfluidic test cassettes or chips that are received within a POC diagnostic instrument and which are able to directly test biological samples that have had no or minimal processing to remove or reduce the effect of PCR inhibitors, as well as being able to deal with a range of sample types also including those harbouring pathogens, by carrying out on-cassette/on-chip processing within the size confines of the cassette/chip.

### BACKGROUND

Point of care (POC) diagnostic systems allow diagnostic testing to occur at or near to the point of care, or the point at which a patient sample is taken i.e. ‘near patient’, rather than requiring samples be sent for central laboratory testing. Whilst initial systems were limited to certain test technologies, more recently, molecular testing methodologies integrated onto cassettes or similar devices have become a realistic point of care option. Such molecular POC systems typically comprise a microfluidic test device, e.g. a cassette, which receives the patient sample and transport it through a fluidic network, and which can carry on-cassette reagents etc., on which the sample is tested; and a diagnostic instrument into which a microfluidic test device is inserted, and which incorporates various means for moving fluid around the cassette, actuators for opening and closing of valves present on the test device, means for actuating movement of materials on the cassette, software for running the test, optics etc.

Molecular point of care (POC) diagnostic systems can carry out molecular diagnostic methods which identify, within a sample, the presence of either host genetic material in the form of DNA or RNA or that of pathogens or other species of interest by detecting or identifying said nucleic acids. Most molecular diagnostic methods require that the nucleic acids of interest be amplified prior to being detected and/or characterised. Polymerase chain reaction (PCR) is a widely used technique used in molecular biology to exponentially amplify a copy, or copies, of a specific segment of DNA to generate a multitude of copies of said DNA sequence. PCR may be used for diagnosis of infectious or hereditary diseases and for genetic analyses in a large variety of sample types. Amplification of DNA by polymerase chain reaction (PCR) for a point of care (POC) diagnostic devices requires reaction mixtures be subjected to repeated rounds of heating and cooling (thermocycling) whist travelling through microfluidic channels present on the test device, which can include holding the reaction mixtures in one or more static chambers present on the cassette. The temperature of the reaction mixture within the cassette therefore must be varied during a PCR cycle. For example, denaturation of DNA typically takes place at greater than 90 degrees, and often close to 98 degrees C. The temperature then needs to drop as annealing a primer to the denatured DNA is typically performed at around 45° C. to 70° C. Finally, the temperature is raised again as the step of extending the annealed primers with a polymerase is typically performed at around 70° C. to 75° C. Various mechanisms to allow for this have been described, with systems employing one or more heating zones on the test device which are aligned with and heated by one or more heaters in the instrument in accordance with a software programme present on the instrument, when the test device is inserted therein. Alternatively, means for rapidly changing the temperature within microfluidic channels may be employed, again the temperature changes are typically managed by on-instrument software. Such mechanisms are known.

Variations of the PCR include reverse transcription (RT-) PCR using RNA as template, which is first transcribed into DNA by a reverse transcriptase, isothermal and/or real-time PCR, which uses fluorescent probes for the detection of the PCR product providing quantitative information. Such variations will be understood to be encompassed by the term “polymerase chain reaction” or “PCR”. It would be understood by those skilled in the art that PCR reagents include reagents such as DNA polymerase, primers (forward and reverse), deoxynucleotide triphosphates (ON-Ms) and PCR buffers, and in the case of reverse transcription (RT-) PCR also reverse transcriptase.

One challenge faced by PCR is that as it is an enzymatic reaction it is therefore sensitive to certain inhibitors that may be present in the sample and the viscosity of the sample to be tested. The aforementioned inhibitors can include larger biological molecules such as proteins, peptides, lipids, metabolites other small molecules and ions. For example, saliva and other biological fluids can contain various inhibitory proteins, including possibly proteinases, a type of enzyme, which can physically degrade the polymerase enzyme used in the PCR reaction. Other PCR inhibitors may bind to the active site of the enzyme or otherwise bind allosterically or can chelate ions required by the polymerase for correct functioning. Inhibitors can include all substances that have a negative effect on the PCR amplification that can originate from the sample itself, added to stabilise the sample, or may be introduced during sample transportation, sample processing e.g. concentration procedures or nucleic acid extraction. PCR inhibition can result in decreased sensitivity or false-negative results. It is therefore important when testing biological samples to ensure that PCR inhibitors are removed or reduced to avoid problems with the test. A number of mechanisms to remove, denature and/or reduce PCR inhibitors are known. These include various extraction methods where the nucleic acid is extracted and, in some cases, concentrated, filtration methods and heat treatment methods. Additives can also be used to degrade, denature, precipitate or otherwise sequester PCR inhibitors. It is therefore common for the sample to be tested to undergo significant processing before the PCR step. As the size of the test device is limited, much of this normally occurs as pre-processing steps prior to the sample being loaded onto/into the test device however it is preferred that such steps be performed on the test device to simplify the user work-flow, speed the time to result, and reduce errors that can occur from manual processing steps.

It is an aim of the present invention to provide relatively cost-effective test devices and methods which allow molecular testing on a POC diagnostic device.

The present invention aims to obviate or mitigate one or more of the limitations of the prior art.

Throughout this document reference to “microfluidic” means with at least one dimension less than 1 millimetre and/or able to deal with microlitre or less portions of fluid.

Throughout this document reference to “cassette” means an assembled unit comprising one or more substrates with channels or chambers therein through which fluid can flow. This can encompass cassettes, plates, chips or similar.

Throughout this document reference to a “network of channels” means one or more channels that may be linear, branched, looped, serpentine or a combination thereof through which fluid, and in particular liquid samples may travel.

The term “zone” refers to a portion of a fluidic channel where a particular activity occurs, or a portion of a fluidic channel with particular, defined characteristics.

The term “fluid communication” refers to a functional connection between two or more areas that allows fluid flow from one of said areas to another of said areas.

The term “flow path” is the route that a liquid sample takes through a microfluidic channel or microfluidic channel network. The flow path need not be linear as the sample may flow back and forth through the channel and via various branch points.

The terms “thermal conditioning”, “thermally conditioned” and similar refer to using an increase in temperature, with or without the addition of other chemicals or biological moieties, to remove or reduce the inhibitory components contained in a sample that will negatively impact PCR and/or to intentionally ‘kill’ or inactivate virus to improve safety.

### SUMMARY OF THE INVENTION

According to an aspect of the present invention there is provided a fluidic test device, which is receivable within a diagnostic instrument for detection of an analyte in a sample; said test device comprising:
a body;
a fluidic channel through the body;
an inlet in fluid communication with the fluidic channel;
means for moving liquid sample through the fluidic channel along a sample flow path for a desired distance and direction; and
a first heating zone;
characterised in that the sample flow path is arranged such that at a first time point a sample will be within at least a portion of the first heating zone and be heated to a thermal conditioning temperature, and at a second time point said sample and one or more polymerase chain reaction reagents will be within the same first heating zone and be heated as part of at least one part of a thermocycling profile.

Advantageously, a single heating zone is used for both a thermal conditioning step to remove or reduce the effect of PCR inhibitors in a sample, and then subsequently the same heating zone is used for at least part of the PCR thermocycling cycle. As the same heating zone is used for two different actions, this allows thermal conditioning of a sample to occur on the test device without taking up significant additional space on the test device. As a consequence, additional heaters and space can be eliminated from the instrument. In turn this allows the test device to accept biological samples with minimal (e.g. dilution only) or no off-chip pre-processing.

Notably, although thermal conditioning can be used to remove or reduce the effect of PCR inhibitors in a sample, it is also able to inactivate certain viruses such as SARS-CoV-2. In the UK, the Advisory Committee on Dangerous Pathogens (ACDP) met in early 2020 to discuss the proposed Hazard Group (HG) for SARS-CoV-2. Whilst SARS-CoV-2 is a novel coronavirus, it was anticipated that the existing safe systems of work for similar HG3 coronaviruses can be used to effectively manage the risks of SARS-CoV-2. Based on the current information, the ACDP committee agreed on a provisional classification of SARS-CoV-2 as a HG3 pathogen. Dangerous pathogens such as SARS-CoV-2 do therefore require very careful handling. In order to increase safety whilst testing samples from positive or suspected positive patients the inactivation of the virus during the testing process provides significant benefits and enables the testing of such pathogens to occur in an appropriately risk assessed point-of-care or near-patient setting.

The sample flow path is arranged such that between the first time point and the second time point said sample will be out with (i.e. not located within) the first heating zone.

Preferably the fluidic channel is a network of fluidic channels. Preferably the network of fluidic channels comprises one or more valves adapted to open and close to form the flow path.

Preferably the means for moving liquid through the fluidic channel allows for predetermined movement of the liquid in a desired direction and fora desired distance along the microfluidic channel.

The first heating zone is adapted to be heated when received within a diagnostic instrument in accordance with programmed protocols. The temperature in the first heating zone is controlled and the first heating zone may be heated or cooled as required.

The first heating zone includes a portion of the microfluidic channel and is associated with a portion of the flow path.

Optionally there is a second heating zone.

Preferably, there is an inhibition reduction reagent in the network of fluidic channels.

Preferably the inhibition reduction reagent is provided in a reagent chamber. The inhibition reduction reagent may be provided as a dried or lyophilised material or in liquid form.

Preferably the inhibition reduction reagent is positioned such that it will mix with the sample in the flow path upstream of the first heating zone.

Preferably the inhibition reduction composition is a reducing agent. Most preferably it is a thiol reducing agent (those skilled in the art would understand this to be a reagent that reduces thiols). Optionally the thiol reducing agent is one or a mixture of dithiothreitol (DTT), 2-Mercaptoethanol (βME), tris(2-carboxyethyl)phosphine (TCEP). TCEP is particularly preferred.

Preferably the fluidic test device is in the form of a microfluidic cassette.

Preferably, the test device further comprises a reducing agent reagent.

According to a second aspect of the present invention there is provided a molecular diagnostic system comprising the fluidic test device of the first aspect, and a diagnostic instrument for receiving said test device, the diagnostic instrument being arranged to receive the fluidic test device in a relatively fixed orientation therein.

Preferably the diagnostic instrument comprises a central processing unit (CPU).

The CPU controls the interactions between the diagnostic instrument and the test device.

Preferably the diagnostic instrument comprises a first heater, said heater arranged to heat the first heating zone of the test device when the test device is received within the diagnostic instrument.

Preferably the diagnostic instrument comprises one or more valve actuators which control the opening and closing of the valves on the microfluidic device.

Preferably the diagnostic instrument comprises one or more movement actuators which control the actuation of the means for moving liquid sample through the fluidic channel on the microfluidic device.

According to third aspect of the present invention there is provided a method for carrying out a polymerase chain reaction on a test device in a molecular diagnostic system, comprising: obtaining a sample;

loading said sample onto the test device of the first aspect via the inlet;

when the test device is inserted into a diagnostic instrument, moving at least an aliquot of sample through the fluidic channel to the first heating zone;

heating the first heating zone to a thermal conditioning temperature to give a thermally conditioned aliquot of sample; combining the thermally conditioned aliquot of sample with one or more PCR reagents;

subjecting the combined aliquot of sample thermally conditioned aliquot of sample and one or more PCR reagents to thermocycling, at least a portion of which occurs in the first heating zone.

Advantageously, the method allows for cost effective on-device molecular identification and/or characterisation of an analyte with minimal or no off-device sample pre-processing to remove or minimise PCR inhibition (other than potentially dilution). Further, by using the same heating zone for both thermal conditioning to remove or reduce the effect of PCR inhibitors in a sample, and then subsequently for at least part of the PCR thermocycling cycle the cassette footprint remains small and cost effective and similarly the diagnostic instrument can be smaller, less complex and more cost effective.

Optionally, after heating the first heating zone to a thermal conditioning temperature to give a thermally conditioned aliquot of sample said thermally conditioned sample is moved to be out with (i.e. not located within) the first heating zone.

Preferably, when heating the first heating zone to a thermal conditioning temperature to give a thermally conditioned aliquot of sample, the aliquot of sample is held in the first heating zone for a predetermined period.

Preferably that period is between 1 and 10 mins.

Most preferably that period is 2 mins.

Preferably the thermal conditioning temperature is between 40° C. and 100° C. (the latter temperature being applicable when there is the ability to pre-pressurise the cassette).

More preferably the thermal conditioning temperature is between 80° C. and 99° C.

Generally, the upper limit is set to prevent boiling of the sample. Higher temperatures require the sample to be held for less time so can be advantageous if looking for rapid processing (although this is balanced against the time it takes to raise the temperature).

More preferably the thermal conditioning temperature is between 90° C. and 99° C.

Most preferably the thermal conditioning temperature is 95° C.

The thermal conditioning temperature is selected to remove or reduce the effect of PCR inhibitors that may be present in the sample, inactivate pathogens present and/or reduce the viscosity. The inhibitors may be present in the sample itself or in upstream components that may be added to the sample during on-chip processing.

Heating the sample can also help to reduce sample viscosity.

Preferably, the step of combining the thermally conditioned aliquot of sample with one or more PCR reagents includes moving the thermally conditioned aliquot of sample to a PCR reagent chamber, combining the thermally conditioned aliquot of sample with the PCR reagents and moving the combined thermally conditioned aliquot of sample and PCR reagents back to the first heating zone.

Preferably, prior to moving at least an aliquot of sample through the network of fluidic channels to a first heating zone, the sample is combined with an inhibition reduction reagent.

Preferably the inhibition reduction reagent is a reducing agent. Most preferably it is a thiol reducing agent. Optionally the thiol reducing agent is tris(2-carboxyethyl)phosphine (TCEP). TCEP is particularly preferred when the sample contains saliva or is from a nasal and/or pharyngeal swab. TCEP is particularly preferred when the polymerase chain reaction is carried out in order to amplify sections of SARS-Cov-2 genetic material as part of a COVID 19 test.

Preferably the molecular diagnostic system is a point of care molecular diagnostic system.

Preferably the molecular diagnostic system is the system of the second aspect.

Various further features and aspects of the invention are defined in the claims.

Unless defined otherwise, all technical and scientific terms used herein have the meaning commonly understood by a person skilled in the art to which this invention belongs.

### BRIEF DESCRIPTION OF THE DRAWINGS

Embodiments of the present invention will now be described by way of example only with reference to the accompanying drawings, where like parts are provided with corresponding reference numerals and in which:

FIG. 1 shows a cross section plan view of a cassette test device according to the present invention, showing the microfluidic channel network within the cassette;

FIG. 2 shows a schematic plan of a cassette with microfluidic channel in accordance with the present invention;

FIG. 3 shows graphs of the raw real-time PCR amplification curves demonstrating the inhibition profile of artificial vaginal fluids with different mucin concentration heated for 5 minutes in a thermal cycler at a range of temperatures. A—2% mucin AVF. B— 1.5% mucin AVF, C— 1% mucin AVF, D—0.5% mucin AVF; and

FIG. 4 shows a perspective view of a diagnostic instrument according to aspects of the present invention.

### DETAILED DESCRIPTION

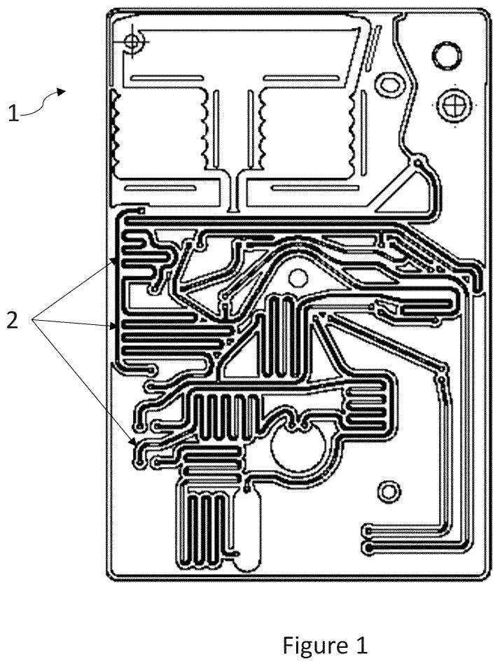

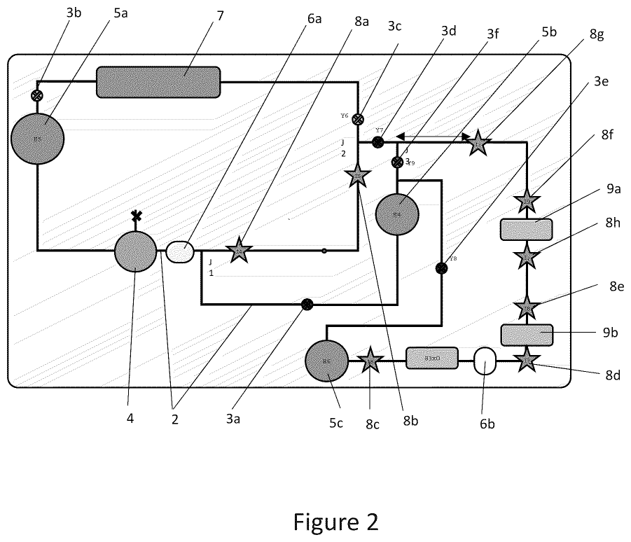

A cross section of a microfluidic cassette 1 showing an internal micro-channel network of microchannels 2 and valves 3 (shown in FIG. 2) is generally depicted in FIG. 1. A schematic diagram showing elements of the cassette is shown in FIG. 2. The micro-channel network of microchannels 2 and valves 3 define a flow path through which a liquid sample can travel. The micro-channel is formed inside the microfluidic cassette, in the desired length and shape to allow the passage of a sample, preferably a biological sample in liquid format, and/or reagents, some or all of which may be incorporated on-cassette during the flow-through, along a fluid flow path and through various zones or areas which allow different activities to occur. Various valves and offshoots can be used to allow mixing, washing, removal and other actions to occur as needed. The channel 2 is formed in a first surface of a first substrate, typically a substantially planar, substantially rigid substrate which in this embodiment is polypropylene. The first substrate is overlaid with a second substrate, which in this embodiment is a polypropylene film. By bonding the first substrate material to the film, for example using laser welding, a substantially closed channel 2 is provided (inlets and outlets can be included as required). It will be understood that if the first substrate is a planar element with an upper and lower surface, the majority of the microchannel can formed in the upper surface or the lower surface. It is however often desirable that the second substrate, i.e. the film, forms the upper wall of the microchannel 2 in use.

Alternatively, it would be understood that although this embodiment has the second substrate as a film, the second substrate can be another material and may itself have grooves or channel formed on its surface that can be aligned with the channels of the first substrate. By bonding the substrates together, a substantially closed channel 2 is provided (again inlets and outlets can be included as required). Other methods for creating channels within the body of a cassette could also be envisaged, e.g. 3D printing.

Where necessary, the first and second substrates can be aligned prior to bonding. The length and cross-sectional shape of the channel 2 can be any appropriate shape to allow for the desired transport and processing of a sample and or reagents. There may be on-cassette reservoirs which fluidly connect to the channel 2 as well as waste chambers or outlets. Discrete portions of the channel 2 can be opened or closed using valves of any appropriate type and fluid, and in particular one or more slugs of liquid sample, can be moved around the cassette using various mechanisms including positive displacement pumps—such microfluidic ‘lab-on-a-chip’ type systems being well known in the art.

It will be understood that the valves and methods for actuating them are known in the art and that one skilled in the manufacture of POC systems would understand the various options available. This is also the case for the means for moving fluid around the network of microchannels, with those skilled in the art being aware of the various options available such as various types of positive displacement pump including bellows pumps, syringe pumps etc.

As shown more clearly in FIG. 2, microfluidic channel 2 has a number of valves 3 that can be actuated to ensure fluid flow to the desired areas of the channel 2 as required. Flow can be in either direction, and whilst the flow path will be described as the path that a sample takes over time, this may involve reciprocating, or bi-directional movement within the microfluidic channel. Directing the flow of material in this manner is known to those skilled in using and making lab-on-a-chip and diagnostic cassette devices.

The cassette 1 is provided with an inlet 4 for receiving a liquid sample into the microfluidic channel 2. Typically, this is biological sample from a human such as sputum, saliva, nasal, pharyngeal swab samples, cervical and/or cervicovaginal swab samples, blood, plasma, cerebro-spinal fluid etc. The inlet is closable with a cap and can be sealed after the sample has been inserted. Notably the present cassette can accept direct samples that have not undergone significant pre-processing to remove inhibitors (other than optionally dilution). A number of compressible bellows 5 are present in fluid communication with the microfluidic channel. The compression and decompression movement of the bellows 5, in combination with the opening and closing of valves 3, moves the sample through the microfluidic channels 2 in a desired direction and distance.

The cassette 1 also has a plurality of reagent chambers 6 (including an anti-inhibition reagent chamber 6a and a PCR reagent chamber 6b) and a waste chamber 7. There are also fluid detection points 8 along the microchannel network of microfluidic films 2 which allow the presence of a fluid sample to be detected. The fluid detection point in this example are formed with at least one surface being a transparent film, which permits the use of emitter/receiver optical sensors in the diagnostic instrument 10 for fluid detection.

The cassette 1 has a PCR zone, which is a designated portion of the cassette where repeated rounds of heating and cooling (thermocycling) occur such that sample travelling through the microchannel network in this zone will, if DNA of interest is present, undergo multiple rounds of denaturation, annealing and extension. The PCR zone will include a number of heating zones 9 which, in use allow portions of the microfluidic network to be heated (and/or cooled). As shown in FIG. 2, there is an annealing and extension heating zone i.e. first heating zone 9a where the temperature can be varied appropriately and a denaturation heating zone i.e. second heating zone 9b (it would however be appreciated by one skilled in the art that a single heating zone could be used for annealing, extension and denaturing if the temperature in the zone is varied appropriately—or there could be three separate heating zones for annealing, extension and denaturing).

In this example, the heating zones are areas of the cassette 1 which, when the cassette 1 is inserted into a diagnostic instrument 10 such as a benchtop or handheld actuator and analyser able to receive the cassette, align with and are brought into close proximity to heaters in the diagnostic instrument 10. Temperatures within the heating zones can be accurately varied. It would however be appreciated that heating elements within the channel or within the cassette could also be utilised.

Importantly, the flow path of the microfluidic network i.e. the route that a liquid sample will travel (which may be linear, but, as in the embodiment shown in FIG. 2, may have reverse flow or reciprocating movement depending on valve positions and actuation of flow direction) is such that the sample will resuspend the anti-inhibition reagent from chamber 6a, pass through a first temperature zone 9, at a first time point before any PCR reagents 6b are collected. This will allow the first temperature zone 9 to be used for thermal conditioning of the sample. The sample and PCR reagents 6b are then mixed and the same temperature zone 9 is used for at least part of the PCR thermocycling. In the example shown in FIG. 2, the first temperature zone 9a is used for both thermal conditioning prior to the addition of PCR reagents and for annealing and extension after the addition of PCR reagents.

The geometry of the microchannels, and in particular of the microchannels that are present in first heating zone 9a are selected such that a liquid sample can be held therein and heated to an appropriate manner. Ideally the sample will be heated in a relatively uniform manner or at least sufficiently so that it is all heated to at least the minimum required temperature without any portions exceeding a higher threshold temperature. It will be appreciated that larger sample volumes may take more time for temperatures to equilibrate and as such and at that point higher temperatures may be detrimental with a significant temperature gradient through the fluid—this can require longer lengths of microchannel in the first heating zone 9a and/or the sample being held in the first heating zone 9a for longer times. It is generally preferred that the sample volume is 500 uL or less (and more preferably 50 uL or less) to give a relatively rapid heating time without requiring a significant length of microchannel in the first heating zone 9a.

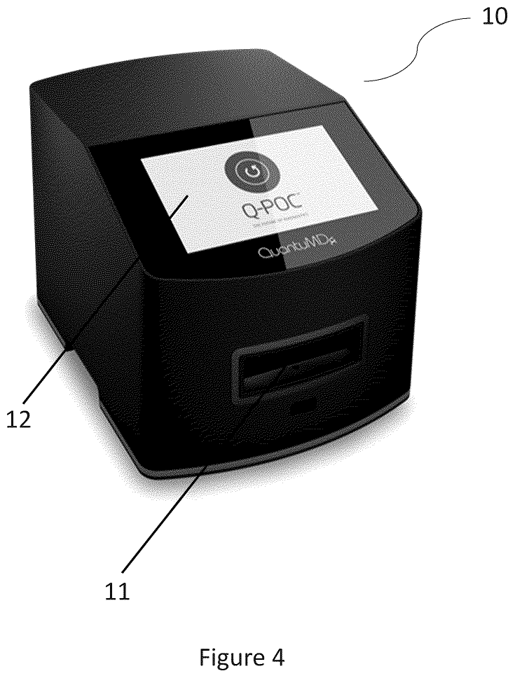

The cassette 1 is able to carry out tests or assays as part of a diagnostic system which also includes a microfluidic diagnostic instrument 10 adapted to receive the cassette 1. A diagnostic instrument 10 is generally depicted in FIG. 4. Such diagnostic instruments are well known in the art and the required components would be known to those skilled in the art of manufacturing molecular POC diagnostic devices. The diagnostic instrument 10 comprises a cassette receiving region 11 that allows the cassette 1 to be inserted into and interact with the diagnostic instrument 10. The receiving region includes a barcode or chip reader that allows information stored on a test cassette to be read and transferred onto the instrument 10. Such information is used to ensure the correct test is carried out by controlling the movement of valve and bellows actuators present in the instrument as well as confirming patient data etc. The diagnostic instrument 10 further comprises components that enable it to interact with the cassette 1 and perform diagnostic tests on a fluid sample contained in the cassette. In the device shown in FIG. 4, the diagnostic instrument 10 also comprises a display screen 12 for displaying data and instructions to a user, and which is also a touchscreen used to receive instructions and information from a user. The diagnostic instrument 10 also comprises one or more diagnostic sensing and/or imaging components for conducting diagnostic sensing and/or imaging on the fluid sample (not shown). The diagnostic instrument 10 also comprises components for heating and/or cooling the fluid sample. Heating elements may be simple cartridge or resistive heaters, Kapton or Kapton-like heaters, or other methods for delivering heat to an area including the use of heated liquids within tubes or channels. Cooling may be achieved through active/passive convection, heat sinks, thermally conductive radiators/fins with or without active movement of ambient or cooled air/liquids. A preferred embodiment uses a peltier heater which can combine both heating and cooling into a single compact unit.

The diagnostic instrument 10 comprises one or more actuators, in this example they are mechanical actuators which physically apply pressure to the outer surface of the bellows valve to compress or remove said pressure to depress, that are able to actuate the bellows present on the cassette. Valve actuators can also be used to open and close valves present on the cassette—again these could be physical but other mechanisms such as Bluetooth™ activated valves could be envisaged. Actuation of the bellows and valves in accordance with pre-programmed instructions allows accurate movement of a sample through the microfluidic network. As indicated above, the initiation of a programme with the correct instructions for a particular test can be initiated either by a user inputting information and/or from reading information present on the test cassette 1. The diagnostic pre-programmed instructions are typically processed by a CPU in the diagnostic instrument.

### Example—Detection of SARS-CoV-2 in Saliva Samples or Nasal and/or Pharyngeal Swab Samples

A novel severe acute respiratory syndrome (SARS)-like coronavirus designated SARS-CoV-2 recently emerged as the causative agent of an infectious disease in humans, COVID-19 exhibiting rapid spread throughout the world.

The present invention could be used to rapidly detect the presence of SARS-CoV-2 in a patient sample using RT-PCR, without the need for significant sample pre-processing. This is particularly relevant as saliva samples or nasal and/or pharyngeal swab samples typically contain a number of inhibitors to RT-PCR so require processing prior to any PCR reaction being performed. As such, the majority of molecular tests based on such sample types are sent for central testing at a laboratory resulting in delays between patients providing samples and results as to whether they have SARS-CoV-2 present in their sample—suggesting they are positive of COVID 19. This can result in challenges with track-and-trace and periods of time where potentially infectious individuals are unclear as to whether or not they have the virus. Given the rapid spread of COVID-19, it being designated as a pandemic by the WHO, the ability to provide POC testing could provide significant benefits.

It is also noted that the advisory committee on dangerous pathogens (ACDP) committee agreed on a provisional classification of SARS-CoV-2 as a HG3 pathogen. Dangerous pathogens such as SARS-CoV-2 require very careful handling. In order to increase safety whilst testing samples from positive or suspected positive patients the inactivation of the virus during the testing process provides significant benefits and enables the testing of such pathogens to occur in an appropriately risk assessed point-of-care or near-patient setting.

Looking at FIG. 2, 400 μl of patient saliva sample is loaded into the inlet 4 of a cassette 1. At this stage, all valves 3 are open. A cap (not shown) is then used to close the inlet with the sample loaded onto the cassette 1. The cassette 1 is loaded into the diagnostic instrument shown in FIG. 4. The cassette 1 is received into the instrument 10 in a fixed orientation such that the optics align with the relevant viewing sections; and the heating elements in the instrument 10 align with (or are proximate to) the first heating zone 9a and the second heating zone 9b present on the cassette 1. A barcode or data chip present on the cassette 1 is read by a receiver in the instrument 10. Any relevant data is input on the screen 12 by the user and the CPU begins the process of actuating the test according to the information provided and the predefined test instructions that it has stored.

Valves 3a, 3b, 3c and 3f are closed. Bellow 5a is compressed by a mechanical actuator in the instrument 10 such that a slug or aliquot of sample moves from the part of the channel 2 near the inlet, as is determined as fluid is detected by fluid detection at point 8a. As the aliquot of sample moves, it passes through reagent chamber 6A which contains anti-inhibition reagent TCEP in lyophilised form. The TCEP is resuspended by the sample. The sample aliquot (now including TCEP) continues to move and is detected by fluid detection point 8b. The inclusion of TCEP is a preferred, but optional, step.

Bellow 5a is further compressed and is detected by fluid detection point 8g, at which point valve 3d is closed. Optionally valve 3c can be opened and the bellow 5a used to send any remaining sample to the waste chamber 7.

A first heater in the instrument 10, which is proximate to heating zone 9a, raises the temperature in the microchannel 2 in heating zone 9a to 95° C.— this is the thermal conditioning temperature. A second heater in the instrument 10 which is proximate heating zone 9b is optionally not turned on at this stage. Valve 3e is closed and valve 3f is opened. Bellows pump 5b is depressed by an actuator until the sample is detected at detection points 8f and 8h. The sample is then held in heating zone 1 for a set period of time—in this case 2 minutes, which is the thermal conditioning time. The thermal conditioning should act to remove or reduce any PCR inhibitors, and/or reduce viscosity and/or inactivate any live virus present in the sample.

The dimensions of the microchannel 2 in the first heating zone 9a is 0.7 mm wide, 0.4 mm deep and 225.42 mm long, when also taking into account the draft and radii, the total volume of this serpentine shaped microchannel, within the first heting zone 9a bounds is 60 uL. A 50u1 slug of liquid sample within this first heating zone would have a length of 187.85 mm.

The heater associated with the first heating zone 9a is then cooled so that the temperature in the microchannel 2 drops to 50° C. The sample can also be allowed to cool with it, for example to below 55° C., prior to the sample being moved, or the sample can be moved before cooling. Bellows 5b and 5c are then actuated to move the now thermally conditioned sample at a controlled rate through reagent chamber 6b which contains PCR reagents until fluid is detected at fluid detection point 8c. The PCR reagents are dried onto a surface of the reagent chamber 6b and are rehydrated by the sample as the sample passes through the chamber.

Further actuation and release of bellows pump 5b and bellows pump 5c moves the sample (now thermally conditioned and containing PCR reagents) back to the first heating zone 9a. At this stage the sample is held in the first heating zone 9a for a period of time to carry out RT (reverse transcription)—it would be understood that a reverse transcription step which is required when looking to identify an RNA virus such as SARS-CoV-2, would not be required for an assay looking to identify or characterise DNA in a sample.

The second heater in the instrument 10 which is proximate, second heating zone 9b, is turned on (although it could also be turned on prior to or during the RT step) and the temperature in the microchannel 2 in the second heating zone 9b is taken to 95° C.— this is the denaturing temperature.

Reciprocating actuation and release of bellows pump 5b and bellows pump 5c then moves the sample back and forth between the first heating zone 9a and the second heating zone 9b. The sample is held in each zone for a set period of time to allow the necessary activity to take place (i.e. for denaturing of the DNA in the second heating zone 9b and for annealing primers and extension in heating zone 9a). The shuttling of the samples between the heating zones is carried out for a set number of cycles (thermocycling). The sample can either be moved to an area where identification or characterisation can occur or as in this example can be monitored during each cycle by passing through a reader portion located in the channel region between fluid sensors 8e and 8f. In this example, known optics are used to detect the generation of fluorescence through the hydrolysis of probes that become attached during the amplification process, however it would be well understood that other methods of detection or characterisation (e.g. sequencing) to determine the presence of analyte in the sample could be used.

Detection of HPV in Cervical and Cervicovaginal Swab Samples Showing that Thermal Conditioning Prior to Direct PCR Reduces Inhibition of the PCR.

Direct PCR has always proved challenging from cervical and cervicovaginal swab samples. This is likely due to the mucin and other glycoproteins found in the mucus which is collected to varying extents with swab-based specimen collection.

Investigation work was carried out to determine whether thermal conditioning of the sample by heating the sample would reduce the inhibition of the PCR. Initially in vitro simulants which mimic the conditions of the vaginal mucus were used. The simulants were given the term artificial vaginal fluid (AVF) and made up as shown in the table below:

| Component name | | |
|---|---|---|
| Died porcine gastric mucin (type III) | 15.0 | g |
| Potassium chloride | 1.752 | g |
| Sodium Chloride | 2.279 | g |
| Sodium acetate | 1.805 | g |
| Albumin | 9.0 | mg |
| Amino acids | 11.0 | mg |
| Glycerol | 0.16 | g |
| Urea | 0.4 | g |
| Lactic acid | 2.0 | g |
| Acetic acid | to pH 4.10 | |
| Demineralised water | to 1 | L |

| | |
|---|---|
| pH | 4.10 |

Alterations were made to the concentration of gastric porcine mucin type III to create samples that would result in a high degree of inhibition. The final mucin content in the simulants were 0%, 0.1%, 0.5%, 1%, 1.5% and 2%.

12.5 μl of each simulant was run in duplicate on a bovine inhibition assay using KAPA3G and D4 PCR buffer resulting in a 50% sample, 50% PCR reagent reaction. For the assay the following were used: Forward Primer: TCTCCCCCATGTTCCTTGAG, Reverse Primer: GGCCCTGTTACTGCCTGTTC, FAM Probe: AGGTCTGAGACTAGGGC. X50 cycles. Annealing: 60° C. Gain 6.8. Threshold: 0.1

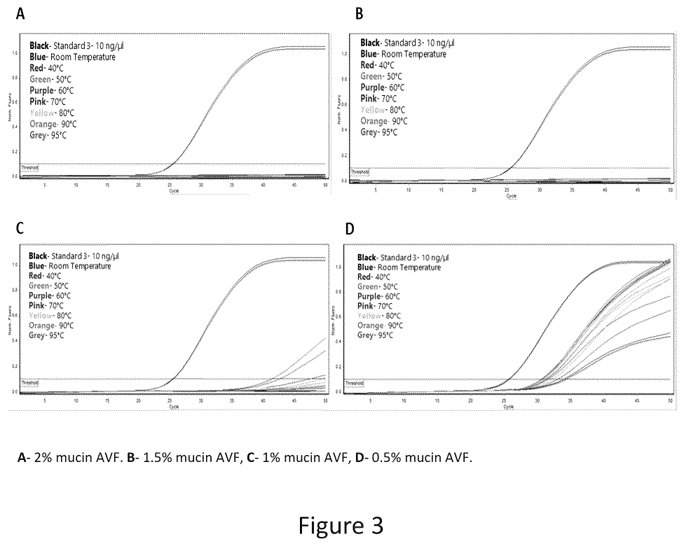

The average CT across the duplicate reactions ±the standard deviation is shown below comparing it to the TE only control which was ran on each PCR and did not contain any AVF. This table directly correlates to the raw amplification curves in FIG. 3.

The Average CT of Artificial Vaginal Fluids with Different Mucin Concentration Heated for 5 Minutes in a Thermal Cycler at a Range of Temperatures.

| | 2% mucin | 1.5% mucin | 1% mucin | 0.5% mucin |
|---|---|---|---|---|
| Temperature | AVF | AVF | AVF | AVF |
| TE only control | 25.46 ± 0.02 | 25.66 ± 0.11 | 25.56 ± 0.06 | 25.86 ± 0.08 |
| Room | 0 | 0 | 0 | 34.22 ± 0.30 |
| Temperature | | | | |
| 40° C. | 0 | 0 | 0 | 32.44 ± 1.03 |
| 50° C. | 0 | 0 | 0 | 31.27 ± 0.25 |
| 60° C. | 0 | 0 | 44.50 ± 4.32 | 30.79 ± 0.02 |
| 70° C. | 0 | 0 | 42.62 ± ND | 30.73 ± 0.11 |
| 80° C. | 0 | 0 | 44.81 ± 0.51 | 30.91 ± 0.10 |
| 90° C. | 0 | 0 | 49.37 ± ND | 31.63 ± 0.98 |
| 95° C. | 0 | 0 | 0 | 31.43 ± 0.04 |

The above data (and FIG. 3) demonstrate that heating between 40-95° C. improves the inhibition observed from the artificial vaginal simulant containing 0.5% mucin (FIG. 3D). Heat does not recover the PCR in the presence of 2% mucin AVF (FIG. 3A) or 1.5% mucin AVF (FIG. 3B), however a signal is detected when the 1% mucin AVF is heated between 60° C. and 90° C. (FIG. 3C). As earlier described, a flocked swab will pick up approximately 200 μl of mucus which when resuspended in 1.2 mL of buffer would result in a final concentration of 0.3-0.8% mucin. Therefore, heating a sample prior to PCR could recover amplification resulting in a signal.

Further experimental work demonstrated that increasing the heating step during the denaturation step does not improve the inhibition observed. This suggests that the heating step must be conducted prior to real-time PCR and not in the presence of the PCR reagents. As such the fluid flow within the diagnostic system and on the diagnostic test cassette of the present invention is arranged such that the heating step for thermal conditioning of a samples is conducted prior to PCR.

All of the features disclosed in this specification (including any accompanying claims, abstract and drawings), and/or all of the steps of any method or process so disclosed, may be combined in any combination, except combinations where at least some of such features and/or steps are mutually exclusive. Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features. The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims are generally intended as “open” terms (e.g., the term “including” or “comprising” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

It will be appreciated that various embodiments of the present disclosure have been described herein for purposes of illustration, and that various modifications may be made without departing from the scope of the present disclosure. Accordingly, the various embodiments disclosed herein are not intended to be limiting, with the true scope being indicated by the following claims.

1. A fluidic test device comprising:
a body;
a fluidic channel through the body;
an inlet in fluid communication with the fluidic channel;
means for moving liquid sample through the fluidic channel along a sample flow path for a desired distance and direction; and
a first heating zone;
wherein the sample flow path is configured such that at a first time point a sample will be within at least a portion of the first heating zone and be heated to a thermal conditioning temperature, and at a second time point said sample and one or more polymerase chain reaction reagents will be within the same first heating zone and be heated as part of at least one part of a thermocycling profile.

2. The fluidic test device as in claim 1, wherein the fluidic channel is a network of fluidic channels comprising one or more valves configured to open and close to form the flow path.

3. The fluidic test device as in claim 1, wherein the means for moving liquid through the fluidic channel allows for predetermined movement of the liquid in a desired direction and for a desired distance along the microfluidic channel.

4. The fluidic test device as in claim 1, wherein the means for moving liquid through the fluidic channel comprises a plurality of positive displacement pumps.

5. The fluidic test device as in claim 1, wherein first heating zone is configured to be heated when received within a diagnostic instrument in accordance with programmed protocols.

6. The fluidic test device as in claim 1, wherein first heating zone includes a portion of the microfluidic channel and is associated with a portion of the flow path.

7. The fluidic test device as in claim 1, wherein there is a second heating zone.

8. The fluidic test device as in claim 1, comprising an inhibition reduction reagent in the fluidic channel.

9. The fluidic test device as in claim 8, wherein the inhibition reduction reagent is provided in a reagent chamber.

10. The fluidic test device as in claim 8, wherein the inhibition reduction reagent is provided as a dried or lyophilized material or in liquid form.

11. The fluidic test device as in claim 8, wherein the inhibition reduction reagent is positioned such that it will mix with the sample in the flow path upstream of the first heating zone.

12. The fluidic test device as in claim 8, wherein the inhibition reduction reagent is a reducing agent.

13. The fluidic test device as in claim 8, wherein the inhibition reduction reagent is a thiol reducing agent.

14. The fluidic test device as in claim 13, wherein the thiol reducing agent is tris(2-carboxyethyl)phosphine (TCEP).

15. The fluidic test device as in claim 1, wherein the fluidic test device is configured as a microfluidic cassette.

16. A molecular diagnostic system comprising the fluidic test device of claim 1, and a diagnostic instrument for receiving said test device, the diagnostic instrument being configured to receive the fluidic test device in a relatively fixed orientation therein.

17. The molecular diagnostic system as in claim 16, wherein the diagnostic instrument comprises a central processing unit (CPU).

18. The molecular diagnostic system as in claim 16, wherein the diagnostic instrument comprises a first heater, said heater configured to heat the first heating zone of the test device when the test device is received within the diagnostic instrument.

19. The molecular diagnostic system as in claim 16, wherein the diagnostic instrument comprises one or more valve actuators configured to control the opening and closing of the valves on the microfluidic device.

20. The molecular diagnostic system as claim 16, wherein the diagnostic instrument comprises one or more movement actuators which control the actuation of the means for moving liquid sample through the fluidic channel on the microfluidic device.

21. A method for carrying out a polymerase chain reaction on a test device in a molecular diagnostic system, the method comprising:
obtaining a sample;
loading said sample onto the fluidic test device of claim 1 via the inlet;
moving at least an aliquot of the sample through the fluidic channel to the first heating zone when the test device is inserted into a diagnostic instrument;
heating the first heating zone to a thermal conditioning temperature to give a thermally conditioned aliquot of sample;
combining the thermally conditioned aliquot of the sample with one or more PCR reagents;
subjecting the combined thermally conditioned aliquot of the sample and one or more PCR reagents to thermocycling, at least a portion of which occurs in the first heating zone.

22. The method for carrying out a polymerase chain reaction as in claim 21, wherein the aliquot of sample is held in the first heating zone for a predetermined period when heating the first heating zone to a thermal conditioning temperature to give a thermally conditioned aliquot of sample.

23. The method for carrying out a polymerase chain reaction as in claim 22, wherein the predetermined period is between 1 and 10 mins.

24. The method for carrying out a polymerase chain reaction as in claim 22, wherein the predetermined period is 2 mins.

25. The method for carrying out a polymerase chain reaction as in claim 21, wherein the thermal conditioning temperature is between 40° C. and 98° C.

26. The method for carrying out a polymerase chain reaction as in claim 21, wherein the thermal conditioning temperature is 95° C.

27. The method for carrying out a polymerase chain reaction as in claim 21, wherein, combining the thermally conditioned aliquot of the sample with one or more PCR reagents comprises moving the thermally conditioned aliquot of the sample to a PCR reagent chamber, combining the thermally conditioned aliquot of the sample with the PCR reagents and moving the combined thermally conditioned aliquot of the sample and PCR reagents back to the first heating zone.

28. The method for carrying out a polymerase chain reaction as in claim 21, wherein the sample is combined with an inhibition reduction reagent prior to moving at least an aliquot of sample through the network of fluidic channels to a first heating zone.

29. The method for carrying out a polymerase chain reaction as in claim 28, wherein the inhibition reduction reagent is a reducing agent.

30. The method for carrying out a polymerase chain reaction as in claim 28, wherein, the inhibition reduction reagent is a thiol reducing agent, which preferably is tris(2-carboxyethyl)phosphine (TCEP).

31. The method for carrying out a polymerase chain reaction as in claim 21, wherein the molecular diagnostic system is a point of care molecular diagnostic system.
